(19) United States
(12) Patent Application Publication
(10) Pub. No.: US 2021/0353754 A1
(43) Pub. Date: Nov. 18, 2021

(54) PIGMENT FOR ANTIMICROBIAL PHOTODYNAMIC THERAPY

(71) Applicant: Christopher John Walinski, Montvale, NJ (US)

(72) Inventor: Christopher John Walinski, Montvale, NJ (US)

(21) Appl. No.: 17/302,774

(22) Filed: May 12, 2021

Related U.S. Application Data
(60) Provisional application No. 62/704,463, filed on May 12, 2020.

(51) Int. Cl. A61K 41/00; A61B 18/20; A61C 5/40; A61C 1/00

(52) U.S. Cl. CPC A61K 41/0028; A61B 18/20; A61B 2018/00577; A61C 1/0046; A61C 5/40

(57) ABSTRACT

A nonstaining photosensitizer includes powdered Nigella sativa seeds in a carrier medium. A method of antimicrobial photodynamic therapy includes administering a photosensitizing composition comprising a Nigella sativa pigment to a treatment site and irradiating the treatment site with a wavelength effective to photoactivate the photosensitizing composition to ablate microorganisms and/or diseased tissue.

### CROSS-REFERENCE TO RELATED APPLICATION

This application claims the benefit of priority of U.S. provisional application No. 62/704,463, filed May 12, 2020, the contents of which are herein incorporated by reference.

### BACKGROUND OF THE INVENTION

The present invention relates to antimicrobial photodynamic therapy and, more particularly, to a pigment composition therefor.

Bacterial infection and inflammation are two of the biggest problems in many areas of health. The use of photosensitizing drugs in combination with light for treating medical conditions is referred to as Photodynamic Therapy (PDT).

Photosensitizers were identified as a potentially effective means of killing microorganisms over 100 years ago (see Moan, et al., “An outline of the hundred-year history of PDT”, Anticancer Res., Sep-Oct 2003, 3591-3600, 23(5A)). A photosensitizer is defined as a material, element, chemical, solution, compound, matter, or substance which is sensitive, reactive, receptive, or responsive to light energy; see U.S. Pat. No. 8,207,211B2. It has been shown that by adding a photosensitizer (e.g., a pigment) to an area susceptible to bacterial infection, certain wavelengths of light and/or lasers have an enhanced effectiveness.

Antimicrobial photodynamic therapy can be useful in treating many medical and dental conditions in a less invasive manner. One of the problems with the photosensitizer products that currently exist is that they stain the tissues with a blue, green, or yellow color. For example, other solutions use toluidine blue, methylene blue and indocyanine green, among others. These traditional compositions are used primarily to stain specimens, so the pigment is quite difficult to remove from soft tissues. Because of the staining nature of these products in a visible area, like the mouth, patients are not as willing to have them used for some situations, such as to treat periodontal disease. Moreover, because existing products are not darkly colored or pigmented, they do not absorb light energy as well as may be desired for optimal bactericidal effectiveness. In other words, they work, but could work better if more highly absorbent of light.

As can be seen, there is a need for a photosensitizer that is highly light absorbent within specified wavelengths and does not stain tissues.

### SUMMARY OF THE INVENTION

The present invention provides a photosensitizer that comprises a plant-based (food-grade and nontoxic) pigment derived from Nigella sativa which is darker than any existing formulation, better absorbs the light wavelengths used to activate the composition, and does not stain tissues. The inventive composition may be effective at killing a broad range of microbes such as Gram positive and Gram-negative bacteria, fungi, and yeast, while not being harmful to the cells of a mammalian subject.

The inventive photosensitizer may be used in any area of medicine where reducing or eliminating bacteria may be helpful, including dental medicine, especially periodontal medicine (e.g., to treat periodontal disease); oncology (e.g., for anti-cancer therapies) as a minimally invasive alternative treatment; surgical medicine; dermatology, esthetic medicine, and veterinary medicine. Specifically, the invention may be used to treat dental conditions including but not limited to the group consisting of: gingivitis, plaque formation, cavity formation, periodontal disease “pockets”, dental caries, root caries, endodontic root canal infection, apical periodontitis, halitosis, and bacterial biofilms that should be eliminated.

In one aspect of the present invention, a nonstaining photosensitizer is provided comprising powdered Nigella sativa seeds in a carrier medium.

In another aspect of the present invention, a method of photodynamic therapy is provided, comprising administering a photosensitizing composition comprising a Nigella sativa pigment to a treatment site; and irradiating the treatment site with a wavelength effective to photoactivate the photosensitizing composition to create an antimicrobial or ablative effect on microorganisms and/or diseased tissue.

These and other features, aspects and advantages of the present invention will become better understood with reference to the following drawings, description, and claims.

### BRIEF DESCRIPTION OF THE DRAWINGS

FIG. 1 is a block diagram of antimicrobial photodynamic therapies in which a composition according to the present invention may be administered;

FIG. 2 is a top plan view illustrating a method of manufacturing a photosensitizing composition according to an embodiment of the present invention;

FIG. 3 is a schematic cross-sectional view of a tooth, illustrating a method of intracanal administration thereof;

FIG. 4 is a schematic cross-sectional view of a tooth and gums, illustrating a method of administration thereof into a periodontal pocket;

FIG. 5 is a perspective view of a tooth prepared for a filling, illustrating a method of intracavity administration thereof; and

FIG. 6 is a front elevation view of an open mouth, illustrating a method of administration thereof orally onto the dorsal surface of a tongue.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

Broadly, one embodiment of the present invention is a photosensitizing composition comprising a pigment derived from Nigella sativa. Additional embodiments include a method of photodynamic therapy utilizing the photosensitizing composition and a kit therefor comprising the photosensitizing composition.

Nigella sativa (N. sativa) is also called black caraway, black seed, black cumin, fennel flower, nigella, nutmeg flower, Roman coriander, and kalonji, and is an annual flowering plant in the family Ranunculaceae, native to the Indian Subcontinent and West Asia.

N. sativa oil may be used as a wetting agent, suspension medium, and/or carrier for the pigment, but the invention is not limited to use of N. sativa oil therefor. For example, in some cases, water or 3% w/v hydrogen peroxide may be used for some applications in which an oil base is undesirable. N. sativa oil contains linoleic acid, oleic acid, palmitic acid, and trans-anethole, and other minor constituents including the aromatics thymoquinone, dihydrothymoquinone (DHTQ), p-cymene, carvacrol, α-thujene, thymol, α-pinene, and β-pinene.

The photosensitizer may be administered by a mode selected from, but not limited to, the group consisting of: subcutaneous, intradermal, oral, intracanal, intracavity, transdermal and topical. Preferably, the composition is locally or topically administered. Subsequent irradiation with visible or infrared light of a suitable wavelength generates singlet oxygen within or on the surface of the bacteria, fungi, or yeast at the treatment site, leading to death of undesirable organisms. Singlet oxygen may be generated by energy transfer from the photosensitizer to dissolved oxygen from surrounding tissues.

In periodontal soft tissues, specifically periodontal pockets created due to periodontal disease, Nigella sativa photosensitizer formulation may be placed within or injected into a pocket adjacent to a tooth and may be irradiated with an appropriate light source to produce photoactivation. Alternatively, after placement of photosensitizer, a higher average power light source may be utilized to remove diseased and infected epithelial tissues lining the periodontal pocket. The photosensitizer may absorb into the target soft tissues, increasing the pigmentation of the diseased tissues and rendering the diseased tissue more susceptible to photodynamic therapy. As a result, a lower light dose may be sufficient to ablate the offending biofilm (potentially including bacteria, fungi, and yeast) and diseased tissues than otherwise may be necessary.

When used to enhance root canal therapy, the paste may be injected into an endodontic canal prior to irradiation. During dental cavity preparation, the paste may be injected into a cavity and irradiated before the cavity is filled. To treat halitosis, the paste may be placed onto the dorsal surface of a tongue before irradiating.

The inventive photosensitizer composition may be prepared as follows. Pure Nigella sativa seeds may be ground into a fine powder. The powder may be thoroughly mixed with pure, unaltered, cold-pressed Nigella sativa oil to an emulsion or paste. The powder and oil may be provided in a ratio of about 1:1 to about 4:1, for example, about a 2:1 ratio. The powder provides a dark color and may be completely black, and the oil may be amber in color. The mixed Nigella sativa oil and powder paste may be placed directly in contact with tissue to be treated. Once combined, the ingredients generally form a dark gray or blackish liquid/gel. The liquid/gel may be placed into a carrier for delivery to the surgical site.

The formulation may comprise ultrafine Nigella sativa powder combined with other ingredients in a carrier medium. The carrier may be selected from the group consisting of a liquid, solution, suspension, emulsion, semi-solid gel, cream, lotion, microemulsion, and a hydrogel. The carrier medium may include a solvent, such as water or 3% (w/v) hydrogen peroxide, which increases the oxidative potential and may improve healing.

In some embodiments, other ingredients may be added to the paste. The formulation may further comprise components selected from the group consisting of an oxygen generator or oxygen carrier; chlorhexidine or salts thereof; anti-inflammatory agents; wound healing enhancing agents; analgesic agents; anesthetics; anti-caries agents; anti-calculus agents; anti-cancer agents; viscosity modifiers (such as a thickening agent); surfactants; flavorants; a diluent; an emulsifier; an antibiotic or other such medicament; a component to improve binding the photosensitizer to the target tissue; and a combination thereof. An oxygen generator may hold peroxide stable within the mixture and may produce enough singlet oxygen or other radical species for anti-microbial benefit. The diluent may be ethanol, water, or pure oil from Nigella sativa seeds. Preferably, the diluent is water.

The Nigella sativa photosensitizer formulation may be administered in multiple doses over an extended period of time with laser or LED irradiation applied after each dose. The total treatment time, including drug-to-light interval and irradiation time, may be about 1 second to about 60 minutes, preferably between about 5 seconds to about 30 minutes. The formulation may be activated by a visible and/or infrared laser and/or LED light source falling within the electromagnetic spectrum from about 380 nm to about 11 μm and may include multiple simultaneous wavelengths. Preferably, the light wavelength provided may be within about the 380 nm to about the 1450 nm range and/or within about the 9.5 μm to about the 11 μm range. The light may be provided as a continuous wave laser or light wave or the light may be pulsed.

The light source may be selected based on the wavelength, beam diameter, exposure time, and/or sensitivity to the cellular and/or acellular organisms. The delivery system may include a wand, light guides, and/or fibers and may include one or more of the following laser types: diode laser; diode pumped solid state laser; diode laser pumped fiber laser; lamp pumped solid state laser with or without a linear converter; CO₂ laser; and solid-state laser or fiber laser doped with ions selected from the group consisting of: Er, Ho, Nd, Tm, Yb, Cr, Ti, and Pr ions. For soft tissue removal in periodontal pockets, an average laser power of about 0.5 W to about 5.0 W may be suitable, more preferably about 1.0 W to about 2.5 W. The light source may have a fluence (PDT) of about 1 J/cm² up to 450 J/cm²; a power density (PDT) of about 1 mW/cm2 to about 1,000 mW/cm2.

Nigella sativa photosensitizer may be included as part of a kit, which may contain photosensitizer solution and one or more components selected from, but not limited to, the group consisting of instructions to user; a photosensitizer mixing dispenser; and a disposable fiber or optical guide.

A method of using the inventive photosensitizer, for example into a periodontal pocket, includes the following. The liquid or gel may be applied directly at the surgical site and left in place for several minutes and then activated using a low power near-infrared laser device. The additional pigmentation at the surgical site increases absorption into the soft tissues by the appropriate wavelength of near-infrared light. The added absorption increases the efficacy and improves the antimicrobial nature of the gel/light combination. The surgical area may be cleared of the liquid or gel using an ultrasonic cleaning device with aerosolized water spray.

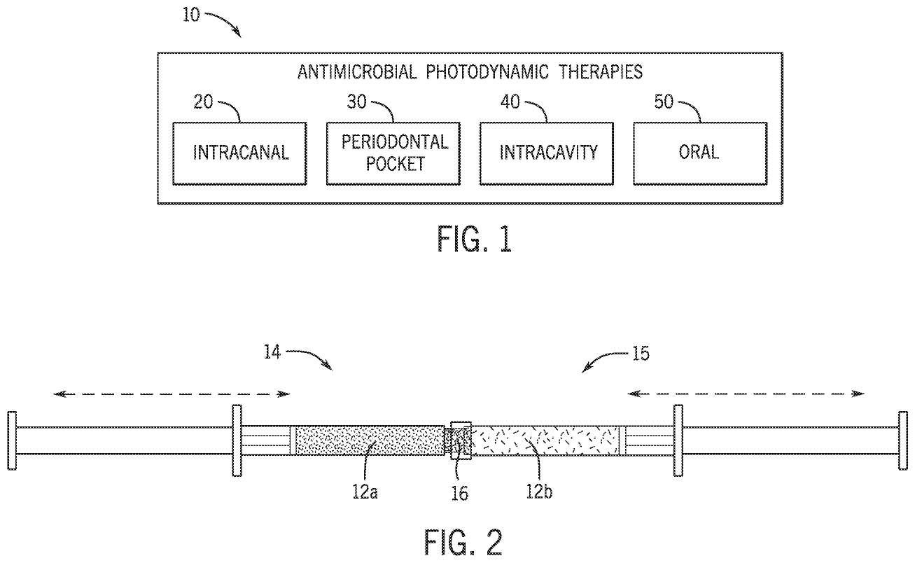

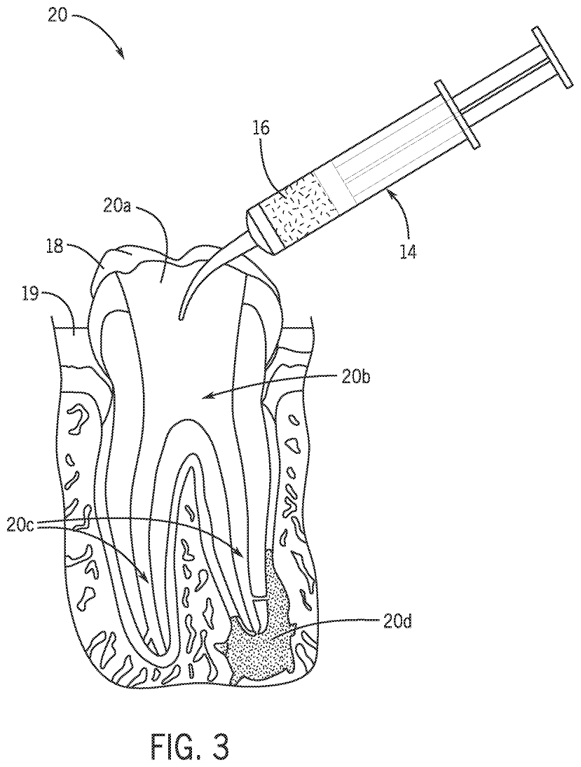

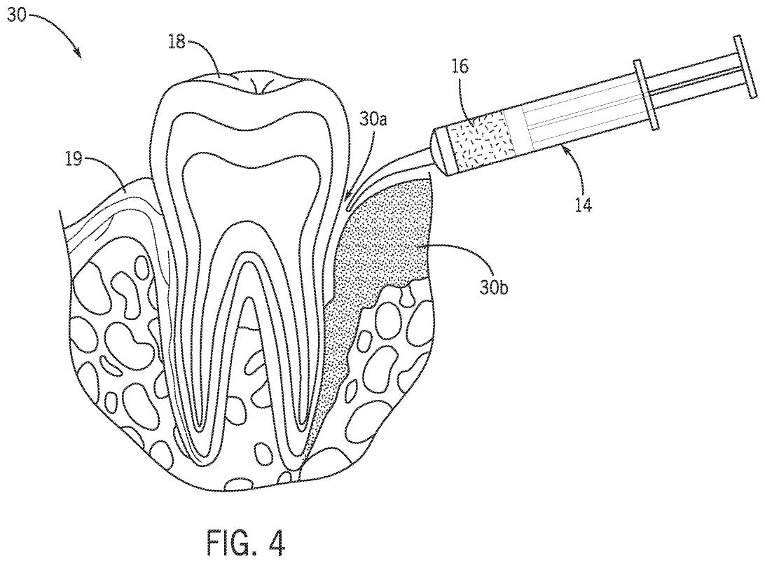

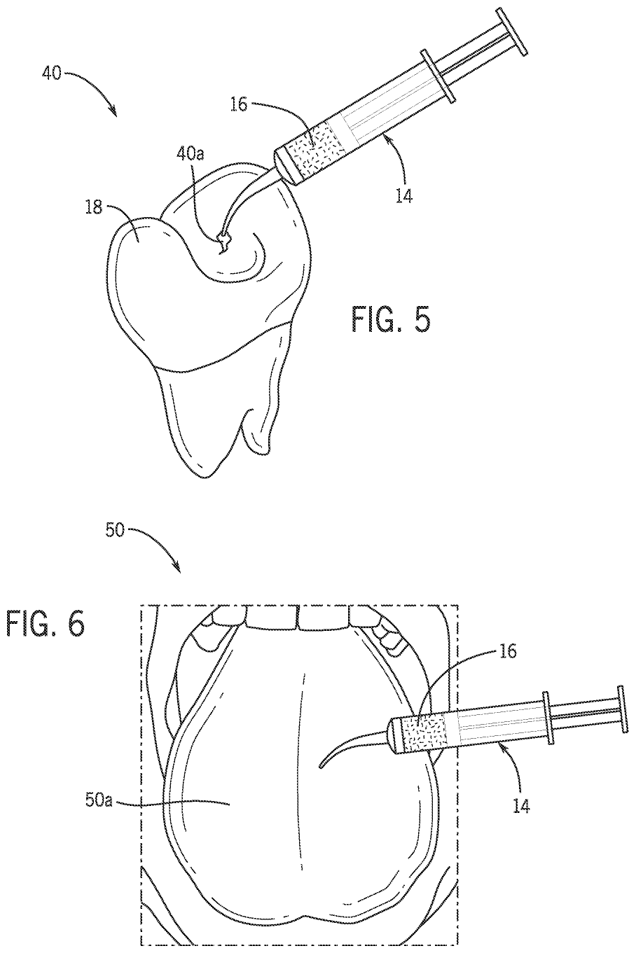

Referring to FIGS. 1 through 6, FIG. 1 illustrates a variety of antimicrobial photodynamic therapies 10 in which the inventive composition may be used, including an intracanal method of administration 20 (shown in FIG. 3), a periodontal pocket method of administration 30 (shown in FIG. 4), a method of intracavity administration 40 (shown in FIG. 5), and a method of oral administration 50 (shown in FIG. 6).

FIG. 2 shows a method of manufacturing a photosensitizing composition according to the present invention. A syringe applicator 14 containing a liquid 12a, such as Nigella sativa oil or 3% w/v hydrogen peroxide, may be coupled to a syringe applicator 15 containing Nigella sativa paste 12b. The contents of the syringes may be moved between the syringes, mixing the liquid 12a with the Nigella sativa paste 12b to produce the inventive photosensitizer product 16.

FIG. 3 illustrates the intracanal method of administration 20 wherein a syringe applicator 14 containing the inventive photosensitizer product 16 is introduced to an access opening 20a into root canals 20c in a tooth 18 pulp chamber 20b to treat an afflicted area 20d adjacent to healthy gums 19. Once the inventive photosensitizer product 16 has been placed, the region may be laser irradiated.

FIG. 4 illustrates the periodontal pocket method of administration 30 wherein a syringe applicator 14 containing the inventive photosensitizer product 16 is introduced to a periodontal pocket 30a formed between a tooth 18 and an inflamed area 30b of the gums 19. Once the inventive photosensitizer product 16 has been placed, the region may be laser irradiated.

FIG. 5 illustrates the method of intracavity administration 40 wherein a syringe applicator 14 containing the inventive photosensitizer product 16 is introduced to a prepared or unprepared cavity 40a within a tooth 18. Once the inventive photosensitizer product 16 has been placed, the region may be laser irradiated. The tooth may be rinsed before chemical preparation for final resin restoration.

FIG. 6 illustrates the method of oral administration 50 wherein a syringe applicator 14 containing the inventive photosensitizer product 16 is introduced to a dorsal surface of a tongue 50a to kill odor-causing bacteria, especially those hidden between the papillae. Once the inventive photosensitizer product 16 has been placed, the region may be laser irradiated. A modified applicator and laser handpiece may be provided for treatment of the tongue.

It should be understood, of course, that the foregoing relates to exemplary embodiments of the invention and that modifications may be made without departing from the spirit and scope of the invention as set forth in the following claims.

What is claimed is:

1. A nonstaining photosensitizer comprising powdered Nigella sativa seeds in a carrier medium.

2. The nonstaining photosensitizer of claim 1, having a formulation selected from the group consisting of a paste, a liquid, a solution, a suspension, an emulsion, a semi-solid gel, a cream, a lotion, a microemulsion, and a hydrogel.

3. The nonstaining photosensitizer of claim 1, further comprising a diluent selected from the group consisting of: ethanol, water, Nigella sativa oil, and 3% w/v hydrogen peroxide.

4. The nonstaining photosensitizer of claim 3, wherein the diluent is Nigella sativa oil and the powdered Nigella sativa seeds to Nigella sativa oil is present in a weight ratio between about 1 to 1 and about 4 to 1.

5. The nonstaining photosensitizer of claim 1, further comprising a component selected from the group consisting of: oxygen generators; oxygen carriers;
chlorhexidine; chlorhexidine salts; anti-inflammatory agents; wound healing enhancing agents; analgesic agents; anesthetics; anti-caries agents; anti-calculus agents; anti-cancer agents; viscosity modifiers; surfactants; flavorants; diluents; emulsifiers; antibiotics; components effective to improve binding the nonstaining photosensitizer to target tissue; and a combination thereof.

6. A method of antimicrobial photodynamic therapy comprising:
administering a photosensitizing composition comprising a Nigella sativa pigment to a treatment site; and
irradiating the treatment site with a wavelength effective to photoactivate the photosensitizing composition to ablate microorganisms and/or diseased tissue.

7. The method of claim 6, wherein the photosensitizing composition is administered by a route selected from the group consisting of: subcutaneous, intradermal, transdermal, and topical.

8. The method of claim 6, wherein the treatment site is a periodontal pocket and the photosensitizing composition is administered by injection.

9. The method of claim 6, wherein the treatment site is an endodontic canal and the photosensitizing composition is administered by injection.

10. The method of claim 6, wherein the treatment site is a cavity and the photosensitizing composition is administered by injection, followed by irradiating the cavity, rinsing the cavity, and filling the cavity.

11. The method of claim 6, wherein the treatment site is a tongue and the photosensitizing composition is administered as a paste applied to a dorsal surface of the tongue.

12. The method of claim 6, wherein the photosensitizing composition is administered in a plurality of doses over a predetermined treatment time.

13. The method of claim 6, wherein a total treatment time, including a drug-to-light interval and an irradiation time, is between about 1 second and about 60 minutes.

14. The method of claim 6, wherein the effective wavelength is between 380 nm and 11 μm.

15. The method of claim 6, wherein the treatment site is irradiated with two or more simultaneous wavelengths.

16. The method of claim 6, wherein irradiating is continuous or pulsed.

17. The method of claim 6, wherein the photosensitizing composition is removed after irradiating the treatment site with an aerosolized water spray.
